(19) United States
(12) Patent Application Publication
(10) Pub. No.: US 2012/0245322 A1
(43) Pub. Date: Sep. 27, 2012

(54) MANUFACTURING LACTIDE FROM LACTIC ACID

(75) Inventors: Chae Hwan Hong, Ansan (KR); Do Suck Han, Seongnam (KR)

(73) Assignee: HYUNDAI MOTOR COMPANY, Seoul (KR)

(21) Appl. No.: 13/307,772

(22) Filed: Nov. 30, 2011

(30) Foreign Application Priority Data
Mar. 25, 2011 (KR) 10-2011-0027122
Jul. 27, 2011 (KR) 10-2011-0074764

(51) Int. Cl. C08G 63/85; B01J 19/00; C07D 319/12

(52) U.S. Cl. 528/357; 549/274; 422/131

(57) ABSTRACT

Disclosed is a method for manufacturing D-lactide from liquid D-lactic acid and a method for manufacturing D-polylactic acid with a weight-average molecular weight 50,000-20,000 g/mol from the prepared D-lactide. The disclosed method is advantageous in that D-lactide can be prepared in high yield through a relatively simple process as compared to the existing method. Thus, the cost for producing D-polylactic acid from the D-lactide can be reduced.

### CROSS-REFERENCE TO RELATED APPLICATIONS

This application claims priority under 35 U.S.C. §119 to Korean Patent Application Nos. 10-2011-0027122, filed on Mar. 25, 2011, and 10-2011-0074764, filed on Jul. 27, 2011, in the Korean Intellectual Property Office, the disclosure of which is incorporated herein by reference in its entirety.

### BACKGROUND

(a) Technical Field

The present invention relates to a method for manufacturing lactide in high yield from D-lactic acid monomers prepared through fermentation.

(b) Background Art

The amazing industrialization since the 20th century appears to be largely based on fossil fuel resources, particularly petroleum. With the rapid industrial development and population growth, the petroleum consumption has been increased continuously as well. Petroleum is an unrenewable resource with limited amount of reserves expected to be exhausted soon. Recently, the carbon dioxide generated by the consumption of fossil fuel has been claimed as one of the main causes of global warming, and researches are striving to improve fuel efficiency for reducing carbon dioxide emission and to reduce dependence on petroleum.

Polymers derived from plants, i.e., biomass polymers, can be prepared by a chemical or biological process from renewable plant resources such as corn, bean, sugar cane, wood, etc. Their value lies in solving the environmental problem through carbon dioxide reduction rather than in biodegradability. Of the biomass polymers, polylactic acid is a carbon neutral, environment-friendly, thermoplastic, linear aliphatic polyester, derived from corn starch or potato starch through fermentation or prepared by polymerizing sugar monomers obtained from saccharification of plant-derived cellulose followed by fermentation.

Despite the various advantages of polylactic acid, however, it seems not suitable for use in automobile parts, because of low impact resistance, low heat deflection temperature, etc., as compared to the petroleum-based chemical polymers. Especially, the low impact strength due to its brittleness delimits its application in automobile parts.

For this reason, industrial application of the polylactic acid resin is restricted because of inferior physical properties when compared to the general-use polymer materials. In particular, for use in automobile engine and chassis parts requiring high heat resistance and impact resistance, improvement of physical properties is essential. As a strategy to solve this problem, the technique of preparing a stereocomplex resin by blending the optical isomers of polylactic acid is often used.

To prepare the stereocomplex resin, techniques for preparing L-polylactic acid and D-polylactic acid are required. At present, L-polylactic acid is commercially available in large scale. But the research for the preparation of D-polylactic acid is still at its early stage. Accordingly, there is a need for the development of a technique allowing the preparation of the D-polylactic acid resin at low cost.

Since lactic acid has one asymmetric carbon atom, it exists as two forms of enantiomers. Meanwhile, since lactide has two asymmetric carbon atoms, there are three stereoisomers, which are L-lactide ((S,S)-lactide), D-lactide ((R,R)-lactide) and meso-lactide ((R,S)-lactide).

L-Lactide and D-lactide are enantiomers of each other. In preparing lactide from lactic acid, it is preferred that the absolute configuration of lactic acid be maintained when it is converted to lactide. Thus, lactide is prepared from oligomeric lactic acid LnA, which results from dehydration of aqueous lactic acid followed by catalytic esterification through back-biting.

Catalysts presented for this reaction include: tin powder, tin halide or tin carboxylate (European Patent Publication Nos. 261,572 and 275,581); tin alkoxide (Great Britain U.S. Pat. No. 1,007,347); and zinc or tin (European Patent Publication No. 264,926 and U.S. Pat. No. 4,797,468).

A process of producing lactide by heating an alkali or alkaline earth metal salt of 2-halopropionic acid in a non-aqueous solvent is described in U.S. Pat. No. 4,727,163, and a process of preparing 1,4-dioxan-2-one and 5-substituted-1,4-dioxan-2-one by contacting carbon monoxide (CO) with formaldehyde, a 1,2-glycol and a catalytic amount of hydrogen fluoride (HF) is described in U.S. Pat. No. 4,070,375.

In U.S. Pat. No. 4,727,163, a block copolymer of a thermally stable polyether core with an α-hydroxy acid (or its ester) is thermally degraded under vacuum conditions to form a cyclic ester. In U.S. Pat. No. 4,835,293, a prepolymer of α-hydroxy acid (or its ester) on a thermally stable core is cracked at or above atmospheric pressure, and cyclic ester vapors formed from the reaction mixture are exited. However, it has not been proved whether the methods disclosed in the above patents are economical and provide good yield. In addition, the related processes are complicated.

### SUMMARY

The present invention relates to a process for preparing cyclic lactide by polymerizing liquid lactic acid into low-molecular-weight polylactic acid and degrading it by depolymerization to induce back-biting in the low-molecular-weight polylactic acid chain. In particular, the present invention provides a process allowing a precise control of degree of polymerization during preparation of liquid lactic acid into low-molecular-weight polylactic acid and depolymerization characteristics during depolymerization, production of lactide from liquid lactic, acid conversion of linear lactic acid dimer and trimer vapor to lactide through catalytic reaction in the presence of alumina.

In one general aspect, the present invention provides a method for manufacturing D-lactide including: (a) converting liquid D-lactic acid to D-polylactic acid of a weight-average molecular weight of 600-1200 g/mol at a temperature of 160-210° C. and a pressure of 10-200 torr; (b) converting the D-polylactic acid of a weight-average molecular weight of 600-1200 g/mol to a gas stream by heating at a temperature of 160-210° C. in the presence of one or more catalyst(s) selected from the group consisting of tin powder, tin halide, tin carboxylate and tin alkoxide; (c) passing the gas stream through a catalyst layer including alumina, silica or an alumina-silica mixture; and (d) separating D-lactide from the gas stream that has passed through the catalyst layer.

In another general aspect, the present invention provides a method for manufacturing D-polylactic acid with a weight-average molecular weight 50,000-20,000 g/mol from the prepared D-lactide.

The above and other aspects and features of the present invention will be described infra.

### BRIEF DESCRIPTION OF THE DRAWINGS

The above and other objects, features and advantages of the present invention will now be described in detail with reference to certain exemplary embodiments thereof illustrated in the accompanying drawings which are given hereinbelow by way of illustration only, and thus are not limitative of the invention, and wherein:

FIG. 1 schematically illustrates a process of preparing lactide and D-polylactic acid through fermentation of D-lactic acid; and

FIG. 2 schematically illustrates a reactor used in Example 2.

### DETAILED DESCRIPTION

Hereinafter, reference will now be made in detail to various embodiments of the present invention, examples of which are illustrated in the accompanying drawings and described below. While the invention will be described in conjunction with exemplary embodiments, it will be understood that the present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The present invention provides a method for manufacturing D-lactide comprising: (a) converting liquid D-lactic acid to D-polylactic acid of a weight-average molecular weight of 600-1200 g/mol at a temperature of 160-210° C. and a pressure of 10-200 torr; (b) converting the D-polylactic acid of a weight-average molecular weight of 600-1200 g/mol to a gas stream by heating at a temperature of 160-210° C. in the presence of one or more catalyst(s) selected from the group consisting of tin powder, tin halide, tin carboxylate and tin alkoxide; (c) passing the gas stream through a catalyst layer comprising alumina, silica or an alumina-silica mixture; and (d) separating D-lactide from the gas stream that has passed through the catalyst layer.

For illustration of the present invention, the structure of lactic acid is defined as follows:

L1A: lactic acid, lactic acid monomer, or 2-hydroxypropionic acid;

LD: lactide, or 3,6-dimethyl-1,4-dioxan-2,5-dione (cyclic);

L2A: lactoyllactic acid, or linear lactic acid dimer;

L3A: lactoyllactoyllactic acid, or linear lactic acid trimer; and

LnA: linear n-oligomer of lactic acid.

The degree of polymerization (DP) of polylactic acid is defined as the number n of lactic acid units covalently linked in the lactic acid polymer.

In step (a) of converting liquid D-lactic acid to D-polylactic acid of a weight-average molecular weight of 600-1200 g/mol at a temperature of 160-210° C. and a pressure of 10-200 torr, liquid D-lactic acid is condensation polymerized under reduced pressure to synthesize low-molecular-weight D-polylactic acid. Specifically, the prepared low-molecular-weight polylactic acid may mainly include L2A and L3A.

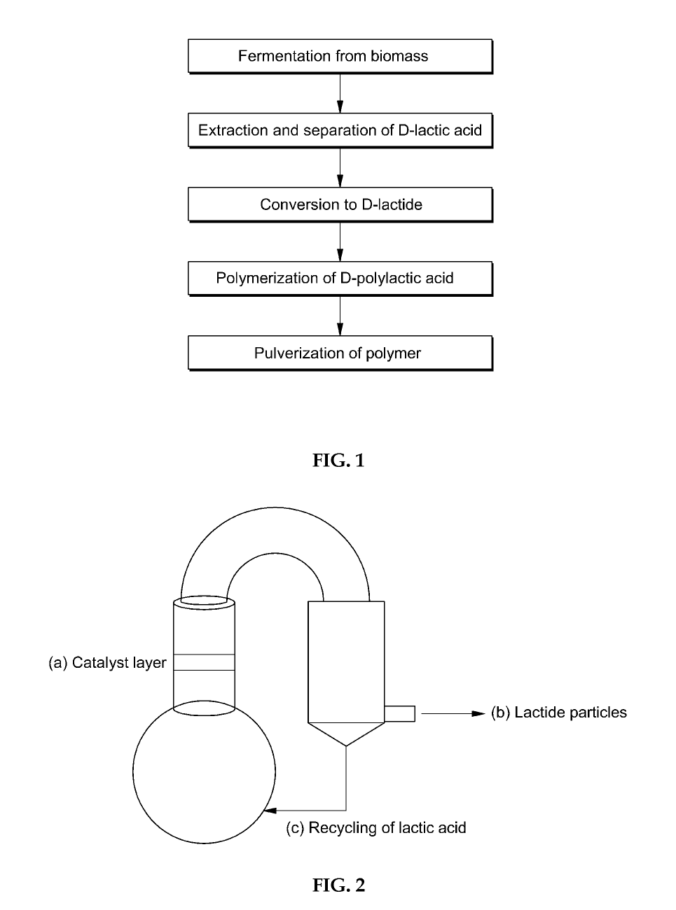

The liquid D-lactic acid may be prepared by saccharifying rice byproduct and starch using α-amylase and amyloglucosidase and then fermenting using Sporolactobacillus inulinus (step 11, FIG. 1).

More specifically, the liquid D-lactic acid may be prepared by fermenting the sugar obtained from the saccharification in a fermentation reactor containing Sporolactobacillus inulinus at 20-50° C. and pH 5-8 for 12-72 hours. Following the fermentation, inorganic matter from the resulting product may be removed by filtration, and the remaining salt of lactic acid (sodium lactate) may be recovered as pure lactic acid by electrodialysis or water-splitting electrodialysis and then concentrated (step 12).

In step (b) of converting the D-polylactic acid of a weight-average molecular weight of 600-1200 g/mol to a gas stream by heating at a temperature of 160-210° C. in the presence of one or more catalyst(s) selected from the group consisting of tin powder, tin halide, tin carboxylate and tin alkoxide, some of the D-polylactic acid is converted to D-lactide while converting unconverted D-polylactic acid to gaseous D-lactide in the presence of one or more catalyst(s) selected from the group consisting of tin powder, tin halide, tin carboxylate and tin alkoxide (step 13) and converting the remaining unconverted polylactic acid into a gas stream at the same time. More specifically, the catalyst may be C₁-C₂₀ tin carboxylate.

The one or more catalyst(s) selected from the group consisting of tin powder, tin halide, tin carboxylate and tin alkoxide may be present in an amount of 0.01-0.5 wt % based on the D-polylactic acid.

In step (c) of passing the gas stream through a catalyst layer comprising alumina, silica or an alumina-silica mixture, the gas stream is passed through a catalyst layer comprising alumina, silica or an alumina-silica mixture to convert D-polylactic acid unconverted in step (b) to D-lactide (again, step 13). In this step, lactic acid is passed through the catalyst layer comprising alumina, silica or an alumina-silica mixture in the form of the gas stream, since lactide yield may decrease significantly when liquid lactic acid reacts with the catalyst.

The gas stream may pass through the catalyst layer being aided by a carrier gas included therein. Specifically, the carrier gas may comprise nitrogen.

More specifically, the catalyst layer comprising alumina, silica or an alumina-silica mixture may comprise 30 wt % or more of alumina, and the catalyst particle size diameter may be 2-6 mm.

In step (d) of separating D-lactide from the gas stream that has passed through the catalyst layer, D-lactide is separated from unconverted D-lactide residue (lactic acid). Specifically, the converted D-lactide may be separated as solid (polymerization, step 14) by cooling to −78 to 10° C., and the residue may be recovered in the form of liquid or gas.

The residue with the D-lactide separated may be returned to the step (a). The thus prepared D-lactide may also be prepared into D-polylactic acid with a weight-average molecular weight 50,000-20,000 g/mol at 150-200° C. using one or more catalyst(s) selected from the group consisting of tin powder, tin halide, tin carboxylate and tin alkoxide and a C₁-C₁₂ alcohol.

### EXAMPLES

The examples and experiments will now be described. The following examples and experiments are for illustrative purposes only and not intended to limit the scope of this invention.

### Preparation Example

### Preparation of D-polylactic acid

Rice bran and crushed rice, byproducts resulting form rice polishing, were grinded into fine powder and mixed with water at a volume ratio of 1:2 to prepare a rice slurry. Then, pH was adjusted to 6.0 using calcium chloride (CaCl₂, available from Tokuyama Co., Japan). Then, after adding α-amylase (20,000 U/cc, available from Wuxi Jieneng Bioengineering, China) as hydrolase to the rice slurry with a dosage of 14 U/g rice byproduct, the mixture was kept at 95° C. for 60 minutes to prepare a first mixture solution, which was then cooled to 60° C. After adding calcium carbonate to the first mixture solution to lower pH to 4.5, amyloglucosidase (available from Wuxi Jieneng Bioengineering, China) was added with a dosage of 110 U/g rice byproduct. The mixture was allowed to react at 60° C. for 30 hours to prepare a second mixture solution. Then, after heating the second mixture solution to 100° C., it was kept at the temperature for 10 minutes in order to deactivate the enzymes. After cooling the second mixture solution to normal temperature, a saccharification solution and a solid sludge residue were separated finally through centrifugation. High-performance liquid chromatography (HPLC) analysis of the separated saccharification solution revealed a sugar concentration of 100 g/L.

Sporolactobacillus inulinus (ATCC1553) was cultured at 40-45° C. for 24-36 hours in a culture medium containing 10.0 g of pancreatic digest of gelatin, 8.0 g of beef extract, 20.0 g of dextrose, 2.0 g of dipotassium phosphate, 1.0 g of Polysorbate 80, 5.0 g of sodium acetate, 2.0 g of ammonium citrate, 0.2 g of magnesium sulfate, and 0.05 g of manganese sulfate in an aqueous solution (1 L).

After adding Sporolactobacillus inulinus to the saccharification solution, fermentation was carried out in a fermentation reactor at 37° C. and pH 6.5 for 72 hours. Then, after removing inorganic matter from the resulting product by filtration, the remaining salt of lactic acid (sodium lactate) was recovered as pure lactic acid by water-splitting electrodialysis and then concentrated. Thus prepared liquid D-lactic acid comprised 61 wt % of L1A, 20 wt % of L2A, 4 wt % of L3A and 15 wt % of water.

### Example 1

The liquid D-lactic acid was polymerized (step 14, FIG. 1) at a temperature of 160° C. and a pressure of 10 torr to prepare D-polylactic acid with a weight-average molecular weight of about 600 g/mol.

### Example 2

FIG. 2 schematically illustrates a reactor 20 used in this example. The reactor had a diameter of about 5 cm and a height of about 5 cm. The reactor was equipped with a transfer line 21 at an upper portion and a catalyst layer 22 of pellet-type alumina (Al₂O₃) with a diameter of 3 mm at a middle portion. A ‘T valve’ 23 was equipped at a lower portion of the reactor to supply D-polylactic acid into the reactor. Further, a nitrogen carrier gas was supplied into the reactor through a line connected to the T valve.

The D-polylactic acid prepared in Example 1 was loaded in the reactor and converted to a gas stream at 220° C. by adding 0.1 wt % of stannous octoate catalyst (([CH₃(CH₂)₃CH(C₂H₅)CO₂]₂Sn), Aldrich). The liquid lactic acid converted to the gas stream was passed through the alumina catalyst layer 22 via the transfer line 21 at the upper portion of the reactor. The gas stream passing through the alumina catalyst layer was cooled to −20° C. to collect solid D-lactide 24 in a cyclone apparatus 25, and the residue 26 was returned to the reactor. The space residence time until the gaseous D-polylactic acid passed through the alumina catalyst layer and reached a receiver vessel 25 was about 1-3 seconds. The yield of produced lactide was calculated on the basis of the quantity of the loaded D-polylactic acid. The result is shown in Table 1.

### Example 3

Lactide was prepared in the same manner as in Example 2, except for using 0.2 wt % of stannous octoate catalyst.

### Comparative Example 1

Lactide was prepared in the same manner as in Example 2, except for not using the stannous octoate catalyst and not using the alumina layer.

### Comparative Example 2

Lactide was prepared in the same manner as in Example 2, except for not using the stannous octoate catalyst.

### Comparative Example 3

Lactide was prepared in the same manner as in Example 2, except for not using the alumina catalyst layer.

TABLE 1

| | Example | | Comparative Example | | |
|---|---|---|---|---|---|
| | 2 | 3 | 1 | 2 | 3 |
| Stannous octoate catalyst (wt %) | 0.1 | 0.2 | — | — | 0.1 |
| Alumina catalyst layer | Yes | Yes | No | Yes | No |
| Yield (%) | 65 | 72 | 15 | 34 | 36 |

As seen from Table 1, use of the stannous octoate catalyst and the alumina catalyst resulted in production of D-lactide particles in high yield from D-polylactic acid.

### Example 4

The D-lactide prepared in Example 2 (3000 g) was put in a reactor equipped with a stirrer and heated to 180° C. under nitrogen flow. Then, stannous octoate (0.9 g) and 1-hexanol (1.8 g) were added. Then, after recovering polymer from the reactor while performing polymerization at 180° C. for 2 hours (step 14, FIG. 1), the polymer was pulverized (Step 15, FIG. 1) to obtain D-polylactic acid with a weight-average molecular weight of about 150,000 g/mol.

The method of the present invention is advantageous in that D-lactide can be prepared in high yield through a relatively simple process as compared to existing methods. Thus, the cost for producing D-polylactic acid from the D-lactide can be reduced.

Furthermore, the absolute configuration of the liquid lactic acid used as the starting material is maintained in the lactide product, unreacted aqueous lactic acid can be recycled, and few byproducts are produced.

The lactide prepared by the method of the present invention can be used as a source material for D-polylactic acid production. A stereocomplex blend comprising the prepared D-polylactic acid and L-polylactic acid has high heat resistance and impact resistance, and may replace the existing petroleum-based polypropylene material with a biomass-derived material. Especially, since it can be used for automobile interior and exterior parts, it can reduce the use of expensive petroleum-based compounds and thus significantly reduce the cost of manufacturing the interior and exterior parts.

The present invention has been described in detail with reference to specific embodiments thereof. However, it will be appreciated by those skilled in the art that various changes and modifications may be made in these embodiments without departing from the principles and spirit of the invention, the scope of which is defined in the appended claims and their equivalents.

What is claimed is:

1. A method for manufacturing D-lactide comprising:
converting liquid D-lactic acid to D-polylactic acid of a weight-average molecular weight of 600-1200 g/mol at a temperature of 160-210° C. and a pressure of 10-200 torr;
converting the D-polylactic acid of a weight-average molecular weight of 600-1200 g/mol to a gas stream by heating at a temperature of 160-210° C. in the presence of one or more catalyst(s) selected from the group consisting of tin powder, tin halide, tin carboxylate and tin alkoxide;
passing the gas stream through a catalyst layer comprising alumina, silica or an alumina-silica mixture; and
separating D-lactide from the gas stream that has passed through the catalyst layer.

2. The method for manufacturing D-lactide according to claim 1, wherein the liquid D-lactic acid is prepared by saccharifying rice byproduct and starch using α-amylase and amyloglucosidase and then fermenting using Sporolactobacillus inulinus.

3. The method for manufacturing D-lactide according to claim 1, wherein the one or more catalyst(s) selected from the group consisting of tin powder, tin halide, tin carboxylate and tin alkoxide is C₁-C₂₀ tin carboxylate.

4. The method for manufacturing D-lactide according to claim 1, wherein the one or more catalyst(s) selected from the group consisting of tin powder, tin halide, tin carboxylate and tin alkoxide is present in an amount of 0.01-0.5 wt % based on the D-polylactic acid.

5. The method for manufacturing D-lactide according to claim 1, wherein the gas stream comprises a carrier gas and the carrier gas comprises nitrogen.

6. The method for manufacturing D-lactide according to claim 1, wherein the catalyst layer comprising alumina, silica or an alumina-silica mixture comprises 30 wt % or more of alumina.

7. The method for manufacturing D-lactide according to claim 1, wherein the catalyst layer comprising alumina, silica or an alumina-silica mixture comprises particles having a diameter of 2-6 mm.

8. The method for manufacturing D-lactide according to claim 1, wherein said separating the D-lactide comprises cooling the gas stream to −78 to 10° C.

9. The method for manufacturing D-lactide according to claim 1, wherein, after said separating, resultant residue is returned to said converting the liquid D-lactic acid to the D-polylactic acid.

10. A method for manufacturing D-polylactic acid with a weight-average molecular weight 50,000-20,000 g/mol, the method comprising:
converting liquid D-lactic acid to D-polylactic acid of a weight-average molecular weight of 600-1200 g/mol at a temperature of 160-210° C. and a pressure of 10-200 torr;
converting the D-polylactic acid of a weight-average molecular weight of 600-1200 g/mol to a gas stream by heating at a temperature of 160-210° C. in the presence of one or more catalyst(s) selected from the group consisting of tin powder, tin halide, tin carboxylate and tin alkoxide;
passing the gas stream through a catalyst layer comprising alumina, silica or an alumina-silica mixture;
separating D-lactide from the gas stream that has passed through the catalyst layer; and
manufacturing the D-polylactic acid from the D-lactide using one or more catalyst(s) selected from the group consisting of tin powder, tin halide, tin carboxylate and tin alkoxide and a C₁-C₁₂ alcohol at a temperature of 150-200° C.

11. A system, comprising:
means for converting liquid D-lactic acid to D-polylactic acid of a weight-average molecular weight of 600-1200 g/mol at a temperature of 160-210° C. and a pressure of 10-200 torr;
means for converting the D-polylactic acid of a weight-average molecular weight of 600-1200 g/mol to a gas stream by heating at a temperature of 160-210° C. in the presence of one or more catalyst(s) selected from the group consisting of tin powder, tin halide, tin carboxylate and tin alkoxide;
means for passing the gas stream through a catalyst layer comprising alumina, silica or an alumina-silica mixture; and
means for separating D-lactide from the gas stream that has passed through the catalyst layer.

12. The system according to claim 11, further comprising:
means for supplying a carrier gas into the gas stream wherein the carrier gas comprises nitrogen.

13. The system according to claim 11, wherein the means for separating the D-lactide comprises means for cooling the gas stream to −78 to 10° C.

14. The system according to claim 11, further comprising:
means for returning resultant residue from said means for separating D-lactide from the gas stream that has passed through the catalyst layer to said means for converting the liquid D-lactic acid to the D-polylactic acid.

15. The system according to claim 11, further comprising:
means for manufacturing D-polylactic acid with a weight-average molecular weight 50,000-20,000 g/mol from the D-lactide using one or more catalyst(s) selected from the group consisting of tin powder, tin halide, tin carboxylate and tin alkoxide and a C₁-C₁₂ alcohol at a temperature of 150-200° C.
